(19) United States
(12) Patent Application Publication
(10) Pub. No.: US 2003/0105596 A1
(43) Pub. Date: Jun. 5, 2003

(54) Methods for evaluating responses of a group of test subjects to a drug or other clinical treatment and for predicting responses in other subjects

(76) Inventors: David Benjamin Goldstein, London (GB); Neil Nathan Bradman, London (GB); Michael Edmund Weale, London (GB)

Correspondence Address: DANN DORFMAN HERRELL & SKILLMAN, SUITE 720, 1601 MARKET STREET, PHILADELPHIA, PA, 19103-2307, US

(21) Appl. No.: 10/280,355

(22) Filed: Oct. 25, 2002

Related U.S. Application Data
(60) Provisional application No. 60/343,420, filed on Oct. 29, 2001.

(51) Int. Cl. G06F 19/00; G01N 33/48; G01N 33/50

(52) U.S. Cl. 702/19

(57) ABSTRACT

Methods are provided which are useful in assessing drug response data, or response data to other types of clinical treatment, in a group of test subjects, and in predicting responses in other subjects, by defining a population genetic structure comprising one or more genetic clusters. The population genetic structure is obtained by analysis of the genotypes of genetic loci present in nucleic acid samples obtained from members of the group of test subjects. The population genetic structure provides an improved tool for assessing the results of a study, such as a clinical trial, into the effectiveness of a drug or other clinical treatment in different sub-populations as it is based directly on genetic criteria for assigning subjects to sub-populations.

### CROSS-REFERENCE TO RELATED APPLICATION

This application claims the benefit of U.S. Provisional Patent Application No. 60/343,420, filed Oct. 29, 2001, the entire disclosure of which is incorporated by reference herein.

### FIELD OF THE INVENTION

The present invention relates to methods for evaluating responses of a group of test subjects to a drug or other clinical treatment and methods for predicting responses in other subjects. In particular, the present invention relates to a method based on deducing a population genetic structure comprising one or more genetic clusters, based on analysis of nucleic acid samples from the members of the group of test subjects.

### BACKGROUND OF THE INVENTION

Many drugs that show therapeutic potential never reach the market because of adverse reactions in some individuals, while other drugs in common use are only effective in a fraction of the population in which they are prescribed. This variation in drug response depends on many factors such as sex, age, and the environment as well as genetic determinants. Since the 1950's pharmacogenetic studies have systematically identified allelic variants at genes that influence drug response, including both drug metabolizing enzymes (DMEs)¹ and drug targets², for example the cytochrome P450 monooxygenase CYP2D^{3,4} and in the N-acetyl transferase NAT2⁵ genes. Detailed functional analysis of variants at genes such as these has clearly demonstrated the importance of genetic variation in drug responses. For example analysis of mutant NAT2 alleles revealed reduction in enzyme activity half life in one case and defective translation leading to reduced enzyme protein in another⁵, while common CYP2D6 variants include a frameshift leading to a truncated non-functional protein and a splice site mutation resulting in the absence of the protein^{3,4}. These and other examples suggest the possibility of genetic tests to predict an individual's response to specific drugs, ultimately allowing medicines to be tailored to specific genetic makeups. Because of the potential commercial and clinical significance of such personalized medicines, understanding the genetic role of variable drug response is one of the primary challenges of biomedical research.

In addition to concerns surrounding individual variation in drug response, however, the geographic structuring of certain variants has focused attention on the possible importance of average differences in drug response across populations. Genetic polymorphisms in DMEs, which are likely to be responsible for much of the phenotypic variation in drug response, vary in frequency among populations², some by as much as twelve-fold¹. For example, the well-known poor metaboliser phenotype of debrisoquine oxidation is due to variant alleles of CYP2D6. Between 5% and 10% of Europeans (but only ˜1% of Japanese) have loss of function variants at this locus which affect the metabolism of more than 40 drugs, including commonly used agents such as β-blockers, codeine and tricyclic antidepressants. CYP2D6 ultra-rapid metaboliser alleles also vary in frequency, even within Europe, from ˜10% in Northern Spain to 1-2% in Sweden⁶. These polymorphisms can lead to acute toxic responses, unwanted drug-drug interactions and also therapeutic failure (e.g. in the case of CYP2D6 duplications)^{1,7}.

These observations make clear that for some drugs the tradeoffs between efficacy and adverse drug reaction will differ not only between individuals but will show average differences in different populations⁸. Thus genetically structured populations may be composed of two or more sub-populations with distinct drug reaction profiles, and thus may be better considered separately in some contexts. This raises the question of the appropriate way to infer human population genetic structure in the context of the evaluation of drug safety and efficacy, and of how to relate this inferred genetic structure to drug response.

### SUMMARY OF THE INVENTION

Broadly, the present invention provides methods which are useful in assessing drug response data, or response data to other types of clinical treatment, in a group of test subjects, and in predicting responses in other subjects, by defining a population genetic structure comprising one or more genetic clusters. The population genetic structure is obtained by analysis of the genotypes of genetic loci present in nucleic acid samples obtained from members of the group of test subjects. The population genetic structure provides an improved tool for assessing the results of a study, such as a clinical trial, into the effectiveness of a drug or other clinical treatment in different sub-populations as it is based directly on genetic criteria for assigning subjects to sub-populations, as compared to criteria used in the prior art such as commonly used ethnic labels. To the extent that inter-individual variation in drug response, or in response to other types of clinical treatment, is a result of genetic differences, a genetic method, implemented properly, is most appropriate for defining sub-populations used to evaluate among-population differences in drug or other clinical treatment response. To the extent that not all genes involved in causing differences in the responses of subjects to a given drug or other clinical treatment are known, a genetic method, implemented properly, is most appropriate for evaluating the importance of unknown or unsuspected genes in causing differences in the responses of subjects to a given drug or other clinical treatment.

Accordingly, in a first aspect, the present invention provides a method for evaluating a property of a clinical treatment in a group of test subjects, the method comprising:

(a) obtaining samples of nucleic acid from members of the group of test subjects;

(b) determining the genotypes at one or more genetic loci present in the samples of nucleic acid from the members of the group of test subjects;

(c) deducing from the genotypes obtained in step (b) a population genetic structure comprising one or more genetic clusters, the clusters comprising the members of the group of test subjects which share common genetic properties;

(d) assigning the members of the group of test subjects to the genetic clusters defined in step (c);

(e) optionally testing to determine whether the population genetic structure comprised of the genetic clusters is consistent, either by adding or removing one or more genetic loci defined in step (b) and then carrying out the procedures defined in steps (c) and (d), or by adding data from one or more other individuals on the genotypes defined in step (b) and then carrying out the procedures defined in steps (c) and (d), or by adding data from one or more other individuals and removing data from one or more test subjects on the genotypes defined in step (b) and then carrying out the procedures defined in steps (c) and (d);

(f) optionally adding or removing test subjects from the group to alter the frequency in the group of different genetic clusters defined in step (c), either for the purpose of ensuring adequate representation of each cluster in the group or for the purpose of reducing or removing the representation of certain clusters;

(g) assigning data on the responses of members of the group of test subjects to the clinical treatment to the genetic clusters to which each member was assigned in step (d);

(h) evaluating the property of the clinical treatment which is the subject of the test by comparing the responses of the different genetic clusters defined in step (c), and optionally comparing the predictive power of these clusters with other ways of categorising the group of test subjects; and

(i) optionally, predicting the responses to the clinical treatment of any other subjects, by carrying out steps (a), (b) and (d) on these other subjects and referring to the property of the clinical treatment in the different genetic clusters as evaluated in step (h).

In a further aspect, the present invention provides a computer program for carrying the method for evaluating a property of a clinical treatment in a group of test subjects.

In a further aspect, the present invention provides a data carrier having a program saved thereon for carrying out the method for evaluating a property of a clinical treatment in a group of test subjects.

In a further aspect, the present invention provides a computer programmed to carry out the method for evaluating a property of a clinical treatment in a group of test subjects.

In a further aspect, the present invention provides a method of providing a population genetic structure for evaluating a property of a clinical treatment in a group of test subjects, the method comprising:

(a) obtaining samples of nucleic acid from members of the group of test subjects;

(b) determining the genotypes at one or more genetic loci present in the samples of nucleic acid from the members of the group of test subjects;

(c) deducing from the genotypes obtained in step (b) a population genetic structure comprising one or more genetic clusters, the clusters comprising the members of the group of test subjects which share common genetic properties;

(d) assigning the members of the group of test subjects to the genetic clusters defined in step (c);

(e) optionally testing to determine whether the population genetic structure comprised of the genetic clusters is consistent, either by adding or removing one or more genetic loci defined in step (b) and then carrying out the procedures defined in steps (c) and (d), or by adding data from one or more other individuals on the genotypes defined in step (b) and then carrying out the procedures defined in steps (c) and (d), or by adding data from one or more other individuals and removing data from one or more test subjects on the genotypes defined in step (b) and then carrying out the procedures defined in steps (c) and (d); and

(f) optionally adding or removing test subjects from the group to alter the frequency in the group of different genetic clusters defined in step (c), either for the purpose of ensuring adequate representation of each cluster in the group or for the purpose of reducing or removing the representation of certain clusters.

In a further aspect, the present invention provides a computer program for carrying the method of providing a population genetic structure for evaluating a property of a clinical treatment in a group of test subjects.

In a further aspect, the present invention provides a data carrier having a program saved thereon for carrying out the method of providing a population genetic structure for evaluating a property of a clinical treatment in a group of test subjects.

In a further aspect, the present invention provides a computer programmed to carry out the method of providing a population genetic structure for evaluating a property of a clinical treatment in a group of test subjects.

By way of example, the nucleic acid samples referred to in step (a) could be obtained from a sample of biological material from an individual such as a blood sample or a buccal swab.

Preferably, the genetic loci referred to in step (b) have a large number of alleles known to exist in humans. For example, the loci could be microsatellite loci.

Preferably, the loci are widely dispersed throughout the human autosomal genome with approximately equal representation on each autosomal chromosome. Preferably, each locus should be separated from all other loci by at least 100 kilobases, to allow a reasonable chance that a large number of recombinations have occurred between all loci on the same chromosome during the course of human population genetic history and thus to increase the chance that any linkage disequilibrium observed among the loci is the result of population structure rather than the result of some other phenomenon. Preferably, each locus should have no known effect on phenotype and should be separated from the nearest known gene or gene-control region by at least 100 kilobases, to allow a reasonable chance that a large number of recombinations have occurred between each locus and other loci likely to be under the influence of selection, which increases the chance that the loci used in step (b) are genetically neutral and thus increases the chance that any linkage disequilibrium or Hardy-Weinberg disequilibrium observed among the loci is the result of population structure rather than the result of some other phenomenon. Preferably, each locus should have a large F_{ST} value as evaluated among populations either selected from around the world or selected to represent relevant important subpopulations vis a vis the potential marketplace of the clinical treatment, to provide a better chance of obtaining well-defined, consistent genetic clusters. Preferably, the number of loci used should have been chosen such that adding more loci does not alter the number or type of genetic clusters deduced in step (c) as evaluated among populations either selected from around the world or selected to represent relevant important subpopulations vis a vis the potential marketplace of the clinical treatment, to provide a better chance that the number of loci used are enough to provide well-defined, consistent genetic clusters.

Conveniently, in step (d), the test subjects can be assigned to the genetic clusters either deterministically, where each subject is assigned wholly to a single genetic cluster, or probabilistically, where each subject is given a probability of membership to each genetic cluster.

By way of example, the population genetic structure referred to in step (c) and the assignment of members referred to in step (d) could be deduced using the STRUCTURE algorithm described in ref. 10. Other tools for deducing genetic structures will be apparent to those skilled in the art.

By way of example, the comparison of the responses of the different genetic clusters referred to in step (h) could be carried out by performing regression analyses using the response of each test subject as the response variable and using the probability of membership of each test subject to each genetic cluster as the explanatory variables. If the response variable was continuous, general linear regression could be applied. If the response variable was dichotomous, logistic regression could be applied. If the response variable was discrete but polychotomous, log-linear regression could be applied. Other ways of categorising the group of test subjects could include using self-identified ethnic labels or functionally-relevant genotypes of genes thought to be important in moderating inter-individual variation in the response to the clinical treatment. Predictive power could be evaluated by comparing the amount of variation in the response variable explained by the different means of categorising the group of test subjects.

For example, step (i) could be achieved by applying the linear models fitted in step (h) to the genetic cluster assignments of other subjects.

In the present invention, “a property of a clinical treatment” includes a test of the effectiveness of the treatment in treating a given condition, a test of the toxicity or side effects of a drug, or a test to optimise an administration protocol of a drug.

As used herein, “the response to the clinical treatment” includes a measurement on a test subject designed to indicate the degree of safety or efficacy of a drug in that individual.

Under step (e), by “consistency”, we mean firstly that the number of clusters deduced in step (c) remains constant, and secondly that the co-membership of subjects to clusters remains constant or, failing this, highly correlated. Under step (e), we also provide for the possibility that population genetic structure is defined by a reference set of individuals that do not form part of the group of test subjects referred to in step (a). The purpose of such a reference set would be to standardise the definition of population genetic structure to allow comparison of results from different groups of test subjects, using the same or different clinical treatments.

Under (f), by “adequate representation” we mean a sample size large enough to evaluate the safety and efficacy of the clinical treatment in a certain cluster.

The evaluation of a property of a clinical treatment might be carried out as part of a clinical trial to investigate the effectiveness of a drug. The evaluation of the property would be in terms not just of the overall safety and efficacy of the drug in the group of test subjects as a whole but also in terms of the sub-populations defined by the genetic clusters deduced as part of this method. This additional information would be valuable for the following reasons. Firstly, if drug response varied greatly in safety and/or efficacy among the genetic clusters, this would provide a warning that the drug may be safely or usefully administered to some people in the target population but not others. This might indicate that targeted marketing of the drug to certain sub-populations would be appropriate, or that the drug was not of sufficiently broad usefulness to be marketed. Secondly, if it was decided that targeted marketing to specific sub-populations was necessary, the method presented here allows for other subjects to be evaluated for their membership of different clusters and based on this a prediction can be made of the response of other subjects which would allow a decision to be made on whether or not to administer the drug or clinical treatment in each individual case. Thirdly, if drug response did not vary greatly in safety and/or efficacy among the genetic clusters, this would indicate that no evidence relating to sub-population specificity was found by this method, increasing the likelihood that the drug could be marketed to a broad target population. Fourthly, if drug response varied greatly in safety and/or efficacy among the genetic clusters and this variation could not be explained by variation in known genes thought to be involved in the drug response that were also genotyped in the group of test subjects, this would indicate that unknown or unsuspected genes were involved in generating inter-individual variation in drug response and thus that further research into these unknown genetic causes could be merited.

Embodiments of the present invention will now be described by way of example and not limitation with reference to the accompanying figures.

### BRIEF DESCRIPTION OF THE FIGURES

FIG. 1. Allele frequencies at each of the DME variants in the STRUCTURE-defined clusters. In all but the last two, black is wild type and white is mutant, for CYP2D6 all mutant alleles are pooled as white, and for NAT2 both tested mutant alleles (*5 and *6) are pooled as white. Several drugs and carcinogens are metabolised by cytochrome P4501A2 (CYP1A2), including the analgesic acetaminophen (Tylenol®). This enzyme is also thought to be involved in the metabolism of antipsychotic drugs¹⁸. Polymorphism in CYP2C19 is responsible for the classical mephenytoin poor metaboliser phenotype but diazepam, barbiturates and antidepressants are also metabolised by this enzyme¹⁹. The classical debrisoquine poor metaboliser phenotype is due to polymorphism in CYP2D6⁷. NAT2 is responsible for the classical isoniazid polymorphism⁵. Quinones are converted to stable hydroquinones by NAD(P):quinone oxidoreductase (DIA4) which also bioactivates antitumour quinones and nitrobenzenes¹⁵. Glutathione-S-transferase mu 1 (GSTM1) conjugates various electrophilic compounds including potent environmental carcinogens such as aflatoxin B₁ epoxides¹. The two NAT2 polymorphisms we genotyped both result in slow acetylator alleles which lead to increased risks of drug toxicity and of certain cancers^{1,5}. Of the CYP2D6 alleles we assayed: CYP2D6*1 is wild type, *3 and *4 have no activity (which can lead to an acute toxic response to some drugs), and *2, *9 and *10 have reduced activity^{17,20}. The CYP1A2 variant genotyped leads to increased enzyme inducibility in smokers²¹. The major polymorphism in CYP2C19 responsible for the mephenytoin poor metaboliser trait was genotyped. After the administration of various drugs this variant can lead to bone marrow toxicity, fatal blood dyscrasias, and other adverse responses¹. Increased susceptibilities to various cancers are associated with the deletion polymorphism in GSTM1 genotyped here, dramatically so for smokers^{1,14}. The mutation in DIA4 leads to a complete absence of the protein and thus loss of the protection against the toxic and carcinogenic effects of quinones¹⁵.

FIG. 2. Allele frequencies at each of the DME variants in the ethnically labelled groups. See FIG. 1 legend for details. A presents Bantu, Ethiopian and Afro-Caribbean frequencies; B those for Norwegians, Ashkenazi Jews and Armenians; and C those for Chinese and New Guineans.

### DETAILED DESCRIPTION

Methods

All subjects were unrelated males. The following X-linked microsatellites were genotyped⁹: DXS984, 996, 1036, 1053, 1062, 1203, 1204, 1205, 1206, 1211, 1212, 1220, 1223, 7103, 8014, 8061, 8068, 8073, 8085, 8086, 8087 and 8099. The following chromosome one microsatellites were genotyped: D1S196, 206, 213, 249, 255, 450, 484, 2667, 2726, 2785, 2797, 2800, 2836, 2842, 2878 and 2890. All form part of the ABI Prism linkage mapping panel 1 and were amplified according to manufacturers instructions. Individuals were assigned into clusters by the program STRUCTURE¹⁰ using the admixture model, with no correlation in allele frequencies among populations and a burn-in time of at least 1 million steps, followed by another million steps of the Markov Chain for data collection. Multiple runs were carried out for each set of conditions to be sure that the chain had converged and in total more than 500 runs were performed. The intronic C734A transversion in CYP1A2 was genotyped by sequencing as were two SNPs in NAT2: C481T defining allele *5 (in complete allelic association with Ile113Thr) and G590A (giving Arg197Gln) defining allele *6. All other alleles were classed as *4 while both mutant allele frequencies were combined for binary analyses. The deletion allele in glutathione-S-transferase mu 1 (GSTM1) was genotyped using GSTM4 amplification as an internal control¹⁴. The C191T transition (giving Pro187Ser) in DIA4¹⁵ and the G117A (leading to a truncated protein) transition in CYP2C19¹⁶ were genotyped by PCR-RFLP. GSTM1 and RFLP amplicons were fluorescently labelled and sizes determined on an ABI 3100 automated sequencer (Applied Biosystems). CYP2D6 SNPs were typed by allele-specific PCR followed by nested reamplification-RFLP detection of the following ‘key’ mutations1⁷: C100T (Pro34Ser) (alleles *10 and *4), G1846A (splicing defect) (allele *4), A2549del (frameshift) (allele *3), 2613-2615AGAdel (Lys281del) (allele *9) and C2850T (Arg296Cys) (allele *2) All other chromosomes were denoted *1 although this will include some non-wild type alleles. CYP2D6*1 was considered to have normal activity, and all other alleles were treated as reduced activity for the binary analyses. CYP2D6 amplicons were labelled using fluorescent primers, then pooled and sized on an ABI 377 automated sequencer (Applied Biosystems). In the case of GSTM1 the assay does not allow the differentiation between homozygous and heterozygous presence of the non-deletion allele. In this case calculations were performed on genotype frequencies, homozygous deletion versus homozygous or heterozygous for the non-deletion allele. DME allele frequencies in the clusters were calculated by distributing an individual's genotype among the clusters according to the proportion of ancestry STRUCTURE determined the individual had in each cluster. When individuals were forced into the cluster in which they had the most ancestry, the results changed very little (not shown). In order to meet the assumption of a multinomial distribution, χ² tables were evaluated after placing individuals into the clusters in which they had most ancestry. We estimated the accuracy of our genotyping by retesting a number of samples from each population. Error rates varied from 0%-7% for the DME SNPs and from 0% to 5% for the microsatellites.

### Population Samples

We genotyped 16 chromosome one microsatellites from the ABI prism panel 1 (an average of 17 cM apart) and 23 X-linked microsatellites (≧2 cM apart)⁹ in each of eight populations: South African Bantu-speakers (46), Amharic and Oromo-speaking Ethiopians from Shewa and Wollo provinces collected in Addis Ababa (48), Ashkenazi Jews (48), Armenians (48), Norwegian-speakers from Oslo (47), Chinese from Sichuan in SW China (39), Papua New Guineans from Madang (48) and Afro-Caribbeans collected in London (30).

### Genetic Structure

A model-based clustering method implemented by the program STRUCTURE¹⁰ was used to assign individuals to sub-clusters on the basis of these genetic data, ignoring their actual population affiliations. This mimics a scenario in which there is cryptic population structure, or no information as to the ethnic origin of the individuals. Briefly, the model implemented in STRUCTURE assumes K clusters, each characterized by a set of allele frequencies at each locus, the admixture model then estimates the proportion of each individuals genome having ancestry in each cluster. We estimated Pr(X|K), where X represents the data, using a model allowing admixture, for K between 1 and 6. From this and a uniform prior on K between 1 and 6, we estimated, Pr(K|X) using Bayes' theorem¹⁰ (Table 1). Virtually all of the posterior probability density is on K=4.

The apportionment of individuals from each of the eight populations into the four STRUCTURE-defined clusters (Table 2) broadly corresponds to four geographical areas: Western Eurasia, Sub-Saharan Africa, China and New Guinea. Importantly 62% of the Ethiopians fall in the first cluster, which encompasses the majority of the Jews, Norwegians, and Armenians, demonstrating that placement of these individuals in a ‘black’ cluster would be an inaccurate reflection of the genetic structure. Only 24% of the Ethiopians are placed in the cluster with the Bantu and most of the Afro-Caribbeans, however 21% of the Afro-Caribbeans are placed in cluster A with the West Eurasians, no doubt reflecting genetic exchange with Europeans. Finally China and New Guinea are placed almost entirely in separate clusters, indicating that the ethnic label Asian is also an inaccurate description of population structure.

Consideration of only the X-linked microsatellites for the purposes of clustering supported K=3, with a very similar clustering to that for the entire data set except that the Chinese and New Guinean clusters were combined into one. When only the chromosome one microsatellites were used the clustering is essentially the same as for the whole data set. This discrepancy may be explained by one of two factors: (i) a lack of resolution in the X chromosome microsatellites, or (ii) a biological factor such as the different number of X chromosomes and autosomes carried by males and females. In order to differentiate these hypotheses we carried out structure runs on the chromosome one data using an equal amount of information to that available from the X chromosome (22 alleles). The chromosome one microsatellites continued to support K=4, indicating that a lack of resolution in the X chromosome microsatellites may not have been the explanation. Perhaps the facts that the X chromosome spends one sixth of the time longer in the female germline than chromosome one does and that females are known to have a higher migration rate than males¹¹ have served to decrease the power of the X-linked loci to detect genetic structure. Smaller random subsets of the loci support a variety of values for K and do not agree on the clustering scheme (not shown). This is likely because there are no natural clusters as there has not been a history of bifurcation in human populations. Our results indicate that a reasonably high number of loci should be used in order that consistency in clustering is achieved. For example, one approach would be to use one marker from each chromosome arm. All analyses we present use the full data set, resulting in four clusters (Table 2).

### Drug Metabolizing Enzymes

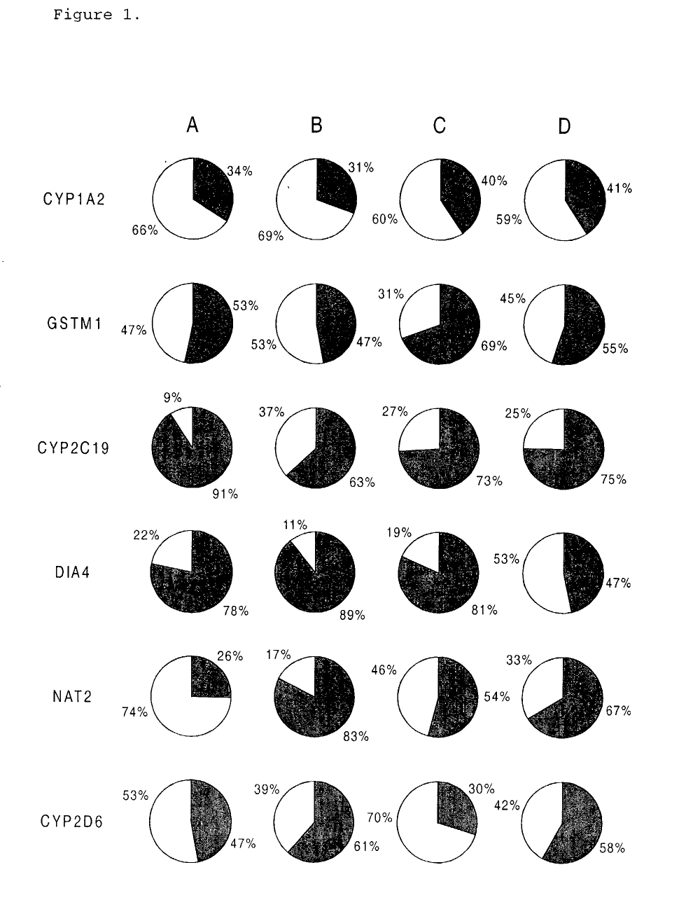

Our selection of DMEs includes representatives of both phase I (oxidation or reduction) and phase II (conjugation) drug metabolism. Three enzymes of the phase I cytochrome P450 family were included: CYP1A2, CYP2C19 and CYP2D6. Three conjugating or phase II metabolism enzymes were also included: NAT2, NAD(P):quinone oxidoreductase (DIA4) and glutathione-S-transferase mu 1 (GSTM1). We determined allele frequencies at eleven variants in these six DMEs, all of which are known to be functionally significant (FIG. 1).

There are notable DME allele frequency differences between the genetically identified clusters (FIG. 1) for five of six reported loci. To assess differentiation across clusters, we counted allele frequencies in each of the clusters and calculated χ²; we also tested for differences in allele frequencies using logistic regression. Using both methods, the allele frequency distributions are highly significantly different for four of the six loci (significant for NAT2, CYP2C19, DIA4 and CYP2D6). The pattern is particularly striking at CYP2C19 where the frequency of the mutant allele (the mephenytoin polymorphism) in cluster B is more than four times that in cluster A (significant at P<0.0001). Extreme differentiation is also evident between clusters B and D for DIA4 where the frequency of the mutant allele (which provides no protection against the toxic effects of quinones) differs by almost five-fold (P<0.0001). This is a noteworthy difference since clusters B and D would be combined as Asian in current drug evaluation using ethnic labels. NAT2 also shows significant differentiation between these two clusters as well as among the others. Strong to modest differences in allele frequencies are observed for the other DMEs between at least two pairs of the clusters in each case. To further explore cluster differentiation, we counted the number of loci for which there are significant allele frequency differences (using χ²) for each of the pairs of clusters. Without correcting for multiple comparisons, this number varied from 2 out of 6 for B vs. D to 5 out of 6 for B vs. C. Given the important differences in drug response determined by these variants, the scope for genetic structuring in drug response is manifestly high. The trade off between therapeutic response and adverse drug reactions will differ between the different clusters identified here thus making it important to perform this kind of genetic analysis to check for such effects in any phase III clinical trial.

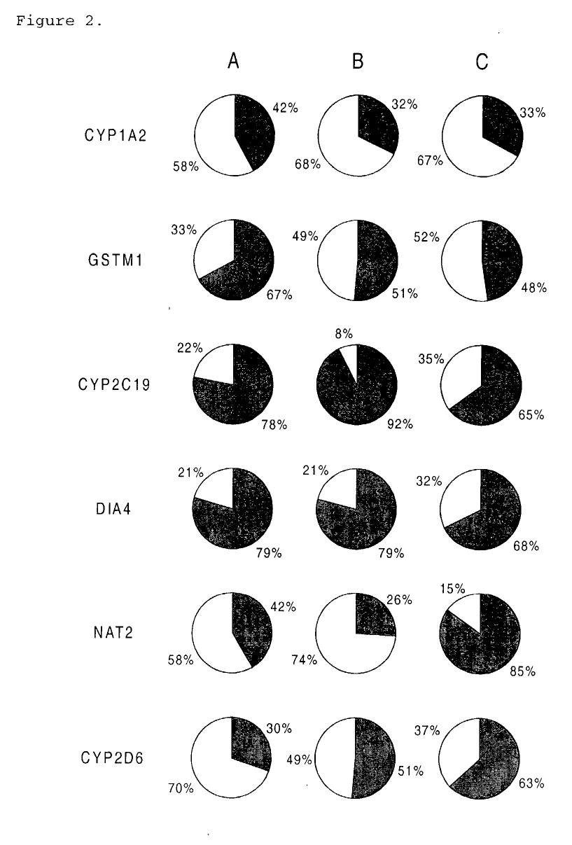

We compared how informative the genetic clusters are versus commonly used ethnic labels by counting the DME allele frequencies in the grouping resulting from commonly used labels: Caucasian (Norwegian, Ashkenazi Jew, Armenian), Black (Bantu, Ethiopian, Afro-Caribbean) and Asian (Chinese, New Guinean) (FIG. 2). The case of DIA4 is noteworthy. The large frequency difference between clusters B and D, driven by the differentiation between China and New Guinea, is averaged when both populations are lumped and so the mutant allele frequency is only one and a half times as high as that in the other two groups. Indeed, the overall differentiation for the ethnic groups is not significant after correction for multiple comparisons. Note that in no case did we observe the reverse in our data. That is, the ethnic labels never show sharp differentiation that is not observed in the clusters. Furthermore, only in the case of CYP2D6 are the allele frequency differentials as high as they are for genetically defined clusters. Thus, although there is some DME allele frequency differentiation between the ethnically labelled groups, in most cases it is less than that seen for the genetic clusters. To demonstrate this formally, we fitted logistic regression models to the allele data using membership in the genetic clusters as the explanatory variables and tested for the increase in goodness of fit obtained by adding the ethnic labels as explanatory variables. We then compared this to the increase in goodness of fit obtained by adding the genetic cluster information to the ethnic group information. Of those DME loci that showed significant differentiation in either the clusters or the ethnic groups (i.e. NAT2, CYP2C19, DIA4 and CYP2D6) in three out of four cases adding genetic cluster information to ethnic labels was more significant than adding ethnic labels to genetic clusters. Only for CYP2D6 was the outcome the reverse.

### Multilocus Interactions

Undesirable drug reactions or interactions may also be due to the possession by an individual of variants at two (or more) loci, as appears to be the case in the increased susceptibility to colorectal cancer in individuals with a rapid/rapid metaboliser phenotype at CYP1A2 and NAT2, greatly so for those who prefer well cooked meat¹². It is therefore important to consider not only allele frequency differences between the inferred clusters, but also frequency differences for multilocus genotypes. There are large frequency differentials between the clusters we have identified for multilocus genotypes which may give rise to phenotypic combinations like this, in fact the frequency of the combination CYP1A2-A/A, NAT2-*4/-observed in cluster B (47%) is more than twice that seen in clusters A (19%) or C (22%; p<0.01 for overall differentiation). When such interactions are important they may be apparent using the genetic analysis described here from the distribution of drug response across inferred clusters.

By carrying out the clustering analysis with the number of clusters set to different values, we can compare the extent of differentiation among the clusters in order to assess the appropriate level of resolution. In the context of a Phase III trial the appropriate benchmark would be in terms of the amount of the total variation in drug response explained by the genetic clusters. A surrogate would be to carry out exact tests of differentiations on relevant functional polymorphisms, stopping when an increase in the number of clusters does not appreciably increase the degree of differentiation. We note, however, that the clustering properties of STRUCTURE can be unstable across different values of K and so implementation of such an analysis using STRUCTURE would not be straightforward.

It is well known that there are inter-ethnic differences in DME allele frequencies and thus in drug response. Our focus here, however, has been to assess the scope for average difference in drug response across genetically inferred clusters. Not only can these be derived in the absence of knowledge about ethnicity (or geographic origin), we also show the inferred clusters are more informative than commonly used ethnic labels. Because of the potential for average difference in drug response to have clinical significance, we conclude that it is not only feasible but a clinical priority to assess genetic structure as a routine part of drug evaluation.

When the most important genes influencing response to a particular drug or group of drugs has been defined, it should be possible to personalize medicine on the basis of an individual's genotype, assuming that routine individual genotyping is commercially and technically feasible. Short of such detailed knowledge, however, it is important to assess whether drugs work similarly in different genetic subgroups. The appropriate level of clustering may be evaluated empirically by assessing the amount of variation in response explained by the inferred clusters. Finally, we have shown that the common ethnic labels currently available to regulatory authorities show a poor correspondence with genetically inferred clusters.

### Analysis of Population Clusters in Biomedical Research

Our implementation of STRUCTURE demonstrates that the familiar ethnic labels are not accurate guides to genetic structure and not as a definitive description of the structure in human populations. Thus, it is advantageous to employ the genetic structures described herein for grouping subjects in clusters for clinical trials, rather than grouping using existing ethnic labels. In employing the approach described in this work, the skilled person should take account of statistical difficulties which may sometimes arise when assessing convergence and the assessment of the appropriate value of K is currently non-rigorous¹⁰. These and other issues can lead to anomalous outcomes, for example, when an implausible value of K is supported in which one of the clusters is more or less empty. Second, results may vary for biological reasons such as when markers are affected differentially by forces acting on the genome, such as gene flow. Detailed analysis of STRUCTURE output and other clustering schemes should thus be explored using a standard battery of markers in a global sample of human populations in order to arrive at a canonical clustering scheme for use in biomedical research. Such an evaluation would need to be geographically exhaustive, and would need to include a sufficient number of markers throughout the genome to ensure that resulting clustering scheme is robust in the sense that similar results would be obtained with different marker and sample sets.

TABLE 1
Inferring the number of clusters.

| K | ln Pr(X\|K) | Pr(K\|X) |
|---|---|---|
| 1 | −33680.97 | ˜0 |
| 2 | −32650.80 | ˜0 |
| 3 | −32046.80 | ˜0 |
| 4 | −31943.23 | 1.000 |
| 5 | −31972.33 | ˜0 |
| 6 | −31987.10 | ˜0 |

TABLE 2
Proportion of membership of each sampled population in
STRUCTURE-defined sub-clusters.

Population

| | A | B | C | D |
|---|---|---|---|---|
| Bantu | 0.04 | 0.02 | 0.93 | 0.02 |
| Ashkenazim | 0.96 | 0.01 | 0.01 | 0.02 |
| Ethiopia | 0.62 | 0.08 | 0.24 | 0.06 |
| Norway | 0.96 | 0.02 | 0.01 | 0.01 |
| Armenia | 0.90 | 0.04 | 0.02 | 0.05 |
| China | 0.09 | 0.05 | 0.01 | 0.84 |
| PNG | 0.02 | 0.95 | 0.01 | 0.02 |
| Afro- | 0.21 | 0.03 | 0.73 | 0.03 |
| Caribbean | | | | |

### REFERENCES

The references cited herein are all expressly incorporated by reference.

1. Weber, W. W. Pharmacogenetics, (Oxford University Press, Oxford, 1997).

2. Evans, W. E. & Relling, M. V. Pharmacogenomics: translating functional genomics into rational therapeutics. Science 286, 487-491 (1999).

3. Gough, A. C. et al. Identification of the primary gene defect at the cytochrome P450 CYP2D locus. Nature 347, 773-776 (1990).

4. Kagimoto, M., Heim, M., Kagimoto, K., Zeugin, T. & Meyer, U. A. Multiple mutations of the human cytochrome P4501ID6 gene (CYP2D6) in poor metabolizers of debrisoquine. Study of the functional significance of individual mutations by expression of chimeric genes. J Biol Chem 265, 17209-17214 (1990).

5. Blum, M., Demierre, A., Grant, D. M., Heim, M. & Meyer, U. A. Molecular mechanism of slow acetylation of drugs and carcinogens in humans. Proc Natl Acad Sci U S A 88, 5237-5241 (1991).

6. Bernal, M. L. et al. Ten percent of North Spanish individuals carry duplicated or triplicated CYP2D6 genes associated with ultrarapid metabolism of debrisoquine. Pharmacogenetics 9, 657-660 (1999).

7. Meyer, U. A. & Zanger, U. M. Molecular mechanisms of genetic polymorphisms of drug metabolism. Annu Rev Pharmacol Toxicol 37, 269-296 (1997).

8. ICH. Ethnic Factors in the Acceptability of Foreign Clinical Data. (International Conference on Harmonisation, 1998).

9. Wilson, J. F. & Goldstein, D. B. Consistent Long-Range Linkage Disequilibrium Generated by Admixture in a Bantu-Semitic Hybrid Population. Am J Hum Genet 67, 926-935 (2000).

10. Pritchard, J. K., Stephens, M. & Donnelly, P. Inference of population structure using multilocus genotype data. Genetics 155, 945-959 (2000).

11. Seielstad, M. T., Minch, E. & Cavalli-Sforza, L. L. Genetic evidence for a higher female migration rate in humans. Nat. Genet. 20, 278-280 (1998).

12. Kohlmeier, L., DeMarini, D. & Piegorsch, W. Gene-nutrient interactions in nutritional epidemiology in Design Concepts in Nutritional Epidemiology (eds. Margetts, B. & Nelson, M.) (Oxford University Press, Oxford, 1997).

13. Raymond, M. & Rousset, F. An exact test for population differentiation. Evolution 49, 1280-1283 (1995).

14. Krajinovic, M., Labuda, D., Richer, C., Karimi, S. & Sinnett, D. Susceptibility to childhood acute lymphoblastic leukemia: influence of CYP1A1, CYP2D6, GSTM1, and GSTT1 genetic polymorphisms. Blood 93, 1496-1501 (1999).

15. Gaedigk, A. et al. NAD(P)H:quinone oxidoreductase: polymorphisms and allele frequencies in Caucasian, Chinese and Canadian Native Indian and Inuit populations. Pharmacogenetics 8, 305-313 (1998).

16. Goldstein, J. A. & Blaisdell, J. Genetic tests which identify the principal defects in CYP2C19 responsible for the polymorphism in mephenytoin metabolism. Methods Enzymol 272, 210-218 (1996).

17. Gaedigk, A. et al. Optimization of cytochrome P4502D6 (CYP2D6) phenotype assignment using a genotyping algorithm based on allele frequency data. Pharmacogenetics 9, 669-682 (1999).

18. Basile, V. S. et al. A functional polymorphism of the cytochrome P4501A2 (CYP1A2) gene: association with tardive dyskinesia in schizophrenia. Mol Psychiatry 5, 410-417 (2000).

19. Ferguson, R. J. et al. A new genetic defect in human CYP2C19: mutation of the initiation codon is responsible for poor metabolism of S-mephenytoin. J Pharmacol Exp Ther 284, 356-361 (1998).

20. Daly, A. K. et al. Nomenclature for human CYP2D6 alleles. Pharmacogenetics 6, 193-201 (1996).

21. Sachse, C., Brockmoller, J., Bauer, S. & Roots, I. Functional significance of a C—&gt;A polymorphism in intron 1 of the cytochrome P450 CYP1A2 gene tested with caffeine. Br J Clin Pharmacol 47, 445-449 (1999).

What is claimed is:

1. A method for evaluating a property of a clinical treatment in a group of test subjects, the method comprising:
(a) obtaining samples of nucleic acid from members of the group of test subjects;
(b) determining the genotypes at at least one genetic loci present in the samples of nucleic acid from the members of the group of test subjects;
(c) deducing from the genotypes obtained in step (b) a population genetic structure comprising at least one genetic cluster, the at least one cluster comprising the members of the group of test subjects which share common genetic properties;
(d) assigning the members of the group of test subjects to the genetic clusters defined in step (c);
(e) optionally testing to determine whether the population genetic structure comprised of the genetic clusters is consistent, either by adding or removing one or more genetic loci defined in step (b) and then carrying out the procedures defined in steps (c) and (d), or by adding data from one or more other individuals on the genotypes defined in step (b) and then carrying out the procedures defined in steps (c) and (d), or by adding data from one or more other individuals and removing data from one or more test subjects on the genotypes defined in step (b) and then carrying out the procedures defined in steps (c) and (d);
(f) optionally adding or removing test subjects from the group to alter the frequency in the group of different genetic clusters defined in step (c), either for the purpose of ensuring adequate representation of each cluster in the group or for the purpose of reducing or removing the representation of certain clusters;
(g) assigning data on the responses of members of the group of test subjects to the clinical treatment to the genetic clusters to which each member was assigned in step (d);
(h) evaluating the property of the clinical treatment which is the subject of the test by comparing the responses of the different genetic clusters defined in step (c), and optionally comparing the predictive power of these clusters with other ways of categorising the group of test subjects; and
(i) optionally, predicting the responses to the clinical treatment of any other subjects, by carrying out steps (a), (b) and (d) on these other subjects and referring to the property of the clinical treatment in the different genetic clusters as evaluated in step (h).

2. The method of claim 1, wherein the nucleic acid samples of step (a) are obtained from a blood sample or a buccal swab from the test subjects.

3. The method of claim 1, wherein the genetic loci of step (b) comprise a plurality of alleles.

4. The method of claim 1, wherein the genetic loci are microsatellite loci.

5. The method of claim 1, wherein test subjects are human and the genetic loci are widely dispersed throughout the autosomal genome of the test subjects, with approximately equal representation on each autosomal chromosome.

6. The method of claim 5, wherein each genetic locus is separated from other loci by at least 100 kilobases.

7. The method of claim 1, wherein each genetic locus has no effect on phenotype of the test subjects.

8. The method of claim 1, wherein the group of test subjects are selected from around the world.

9. The method of claim 1, wherein the group of test subjects are selected to represent subpopulations of subjects relevant to the clinical treatment being assessed.

10. The method of claim 1, wherein the number of loci is chosen such that adding more loci does not alter the number or type of genetic clusters deduced in step (c).

11. The method of claim 1, wherein in step (d), the test subjects are assigned to the genetic clusters deterministically, where each test subject is assigned wholly to a single genetic cluster.

12. The method of claim 1, wherein in step (d), the test subjects are assigned to the genetic clusters probabilistically, where each test subject is given a probability of membership to each genetic cluster.

13. The method of claim 1, wherein the population genetic structure referred to in step (c) and the assignment of members referred to in step (d) are deducible using the STRUCTURE algorithm.

14. The method of claim 1, wherein the comparison of the responses of the different genetic clusters in step (h) is carried out by performing regression analyses using the response of each test subject as a response variable and using the probability of membership of each test subject to each genetic cluster as explanatory variables.

15. The method of claim 1, wherein when the response variable is continuous, general linear regression is applied.

16. The method of claim 1, wherein when the response variable is dichotomous, logistic regression is applied.

17. The method of claim 1, wherein when the response variable is discrete but polychotomous, log-linear regression is applied.

18. The method of claim 1, wherein the test subjects are categorised using self-identified ethnic labels or functionally-relevant genotypes of genes involved in moderating inter-individual variation in the response to the clinical treatment.

19. The method of claim 1, wherein step (i) is achieved by applying the linear models fitted in step (h) to the genetic cluster assignments of other subjects.

20. The method of claim 1, wherein the property of a clinical treatment is selected from the group consisting of a test of the effectiveness of the treatment in treating a given condition, a test of the toxicity or side effects of a drug, and a test to optimise an administration protocol of a drug.

21. The method of claim 1, wherein the response to the clinical treatment is a measurement on a test subject designed to indicate the degree of safety or efficacy of a drug in that individual.

22. The method of claim 1, wherein the evaluation of a property of a clinical treatment is part of a clinical trial to investigate the effectiveness of a drug.

23. The method of claim 1, wherein the evaluation of the property of a clinical treatment comprises determining the overall safety and efficacy of the drug in the group of sub-populations defined by the genetic clusters.

24. A computer program for carrying out the method for evaluating a property of a clinical treatment in a group of test subjects as defined in claim 1.

25. A data carrier having a program saved thereon for carrying out the method for evaluating a property of a clinical treatment in a group of test subjects as defined in claim 1.

26. A computer programmed to carry out the method for evaluating a property of a clinical treatment in a group of test subjects as defined in claim 1.

27. A method of providing a population genetic structure for evaluating a property of a clinical treatment in a group of test subjects, the method comprising:
(a) obtaining samples of nucleic acid from members of the group of test subjects;
(b) determining the genotypes at at least one genetic loci present in the samples of nucleic acid from the members of the group of test subjects;
(c) deducing from the genotypes obtained in step (b) a population genetic structure comprising at least one genetic clusters, the clusters comprising the members of the group of test subjects which share common genetic properties;
(d) assigning the members of the group of test subjects to the genetic clusters defined in step (c);
(e) optionally testing to determine whether the population genetic structure comprised of the genetic clusters is consistent, either by adding or removing one or more genetic loci defined in step (b) and then carrying out the procedures defined in steps (c) and (d), or by adding data from one or more other individuals on the genotypes defined in step (b) and then carrying out the procedures defined in steps (c) and (d), or by adding data from one or more other individuals and removing data from one or more test subjects on the genotypes defined in step (b) and then carrying out the procedures defined in steps (c) and (d); and
(f) optionally adding or removing test subjects from the group to alter the frequency in the group of different genetic clusters defined in step (c), either for the purpose of ensuring adequate representation of each cluster in the group or for the purpose of reducing or removing the representation of certain clusters.

28. A computer program for carrying out the method of providing a population genetic structure for evaluating a property of a clinical treatment in a group of test subjects as defined in claim 27.

29. A data carrier having a program saved thereon for carrying out the method of providing a population genetic structure for evaluating a property of a clinical treatment in a group of test subjects as defined in claim 27.

30. A computer programmed to carry out the method of providing a population genetic structure for evaluating a property of a clinical treatment in a group of test subjects as defined in claim 27.
